(19) United States
(12) Patent Application Publication
(10) Pub. No.: US 2012/0184982 A1
(43) Pub. Date: Jul. 19, 2012

(54) Methods and Systems for Treating Aneurysms

(75) Inventors: Steve Herbowy, Palo Alto, CA (US); Jean-Pierre Dueri, Los Gatos, CA (US); Jason Fox, San Carlos, CA (US)

(73) Assignee: Endologix, Inc., Irvine, CA (US)

(21) Appl. No.: 13/354,094

(22) Filed: Jan. 19, 2012

Related U.S. Application Data
(60) Provisional application No. 61/434,290, filed on Jan. 19, 2011.

(51) Int. Cl. A61M 29/00

(52) U.S. Cl. 606/194

(57) ABSTRACT

A system for treating an aneurysm comprises an elongate flexible shaft and a nosecone. An expandable member may be used to expand an expandable endoframe within a double-walled filling structure. The filling structure is adapted to be filled with a hardenable fluid filing medium so that an outer wall conforms to an inside surface of the aneurysm and an inner wall forms a substantially tubular lumen to provide a path for blood flow. In the expanded configuration the endoframe engages the inner wall of the filling structure. One or more tethers releasably couple the filling structure and/or endoframe with the shaft thereby constraining axial movement of the structures relative to each other. The nosecone is constructed to have a series of side ports and a through lumen such that the nosecone can be positioned with a guidewire and simultaneously receive a contrast media for performing angiography through the lumen.

### CROSS-REFERENCES TO RELATED APPLICATIONS

The present application is a non-provisional of, and claims the benefit of U.S. Provisional Patent Application No. 61/434,290, filed Jan. 19, 2011, the entire contents of which are incorporated herein by reference.

The present application is related to U.S. patent application Ser. No. 12/429,474, filed on Apr. 24, 2009, the full disclosure of which is incorporated herein by reference.

### BACKGROUND OF THE INVENTION

1. Field of the Invention

The present invention relates generally to medical systems and methods for treatment. More particularly, the present invention relates to systems and methods for treating aneurysms.

Aneurysms are enlargements or “bulges” in blood vessels which are often prone to rupture and which therefore present a serious risk to the patient. Aneurysms may occur in any blood vessel but are of particular concern when they occur in the cerebral vasculature or the patient's aorta.

The present invention is particularly concerned with aneurysms occurring in the aorta, particularly those referred to as aortic aneurysms. Abdominal aortic aneurysms (AAA's) are classified based on their location within the aorta as well as their shape and complexity. Aneurysms which are found below the renal arteries are referred to as infrarenal abdominal aortic aneurysms. Suprarenal abdominal aortic aneurysms occur above the renal arteries, while thoracic aortic aneurysms (TAA's) occur in the ascending, transverse, or descending part of the upper aorta.

Infrarenal aneurysms are the most common, representing about eighty percent (80%) of all aortic aneurysms. Suprarenal aneurysms are less common, representing about 20% of the aortic aneurysms. Thoracic aortic aneurysms are the least common and often the most difficult to treat.

The most common form of aneurysm is “fusiform,” where the enlargement extends about the entire aortic circumference. Less commonly, the aneurysms may be characterized by a bulge on one side of the blood vessel attached at a narrow neck. Thoracic aortic aneurysms are often dissecting aneurysms caused by hemorrhagic separation in the aortic wall, usually within the medial layer. The most common treatment for each of these types and forms of aneurysm is open surgical repair. Open surgical repair is quite successful in patients who are otherwise reasonably healthy and free from significant co-morbidities. Such open surgical procedures are problematic, however, since access to the abdominal and thoracic aortas is difficult to obtain and because the aorta must be clamped off, placing significant strain on the patient's heart.

Over the past decade, endoluminal grafts have come into widespread use for the treatment of aortic aneurysm in patients who cannot undergo open surgical procedures. In general, endoluminal repairs access the aneurysm “endoluminally” through either or both iliac arteries in the groin. The grafts, which typically have been fabric or membrane tubes supported and attached by various endoframe structures, are then implanted, typically requiring several pieces or modules to be assembled in situ. Successful endoluminal procedures have a much shorter recovery period than open surgical procedures.

Present endoluminal aortic aneurysm repairs, however, suffer from a number of limitations. For example, a significant number of endoluminal repair patients experience leakage at the proximal juncture (attachment point closest to the heart) within two years of the initial repair procedure. While such leaks can often be fixed by further endoluminal procedures, the need to have such follow-up treatments significantly increases cost and is certainly undesirable for the patient. A less common but more serious problem has been graft migration. In instances where the graft migrates or slips from its intended position, open surgical repair is required. This is a particular problem since the patients receiving the endoluminal grafts are often those who are not considered to be good surgical candidates.

Further shortcomings of the present endoluminal graft systems relate to both deployment and configuration. For example, many of the commercially available endovascular systems are too large (above 12 F) for percutaneous introduction. Moreover, current devices often have an annular support frame that is stiff and difficult to deliver as well as unsuitable for treating many geometrically complex aneurysms, particularly infrarenal aneurysms with little space between the renal arteries and the upper end of the aneurysm, referred to as short-neck or no-neck aneurysms. Aneurysms having torturous geometries are also difficult to treat. Although treatment devices exist for excluding the aneurysms, it can be difficult to visualize the aneurysm, the device, and the flow of blood during deployment to ensure proper sealing and placement of the treatment device.

For these reasons, it would be desirable to provide improved methods and systems for the endoluminal and minimally invasive treatment of aortic aneurysms. In particular, it would be desirable to provide systems having lower delivery profile and methods which can be delivered percutaneously and that can track and be deployed in tortuous vessels. It would also be desirable to provide prostheses with minimal or no endoleaks, which resist migration, which are flexible and relatively easy to deploy, and which can treat many if not all aneurismal configurations, including short-neck and no-neck aneurysms as well as those with highly irregular and asymmetric geometries. Additionally, there is a need for such systems and methods which are compatible with current designs for endoluminal endoframes and grafts, including single lumen endoframes and grafts, bifurcated endoframes and grafts, parallel endoframes and grafts, as well as with double-walled filling structures which are the subject of the commonly owned, copending applications described below. It would also be desirable to provide systems and methods that provide feedback to the operator as to the positioning and deployment of the endoluminal repair device in the aneurysm and allow the operator to visualize the morphology of the aneurysm as well as the flow of blood through the device or aneurysm. The systems and methods would preferably be deployable with the endoframes and grafts at the time the endoframes and grafts are initially placed. At least some of these objectives will be met by the inventions described hereinbelow.

2. Description of the Background Art

U.S. Patent Publication No. 2006/0025853 describes a double-walled filling structure for treating aortic and other aneurysms. Copending, commonly owned U.S. Patent Publication No. 2006/0212112, describes the use of liners and extenders to anchor and seal such double-walled filling structures within the aorta. The full disclosures of both these publications are incorporated herein by reference. PCT Publication No. WO 01/21108 describes expandable implants attached to a central graft for filling aortic aneurysms. See also U.S. Pat. Nos. 5,330,528; 5,534,024; 5,843,160; 6,168,592; 6,190,402; 6,312,462; 6,312,463; U.S. Patent Publications 2002/0045848; 2003/0014075; 2004/0204755; 2005/0004660; and PCT Publication No. WO 02/102282.

### BRIEF SUMMARY OF THE INVENTION

The present invention relates generally to medical devices and methods, and more specifically to systems and methods for the treatment of aneurysms, particularly aortic aneurysms including both abdominal aortic aneurysms (AAA) and thoracic aortic aneurysms (TAA). The systems may be introduced percutaneously or by surgical cutdown into a patient and may have an outer diameter ranging preferably from 10 French to 18 French and more preferably from 12 French to 16 French.

In a first aspect of the present invention, a system for treating an aneurysm in a blood vessel comprises an elongate flexible shaft having a proximal region and a distal region. The system may include an endograft, such as a filling structure, for excluding the aneurysm and a fenestrated nosecone having a through lumen for both advancement of the shaft along a guidewire and for simultaneously performing angiography. The endograft may comprise a first double-walled filling structure, having an outer and inner wall, the filling structure disposed over the distal region of the shaft. The filling structure may be filled with a hardenable fluid filing medium so that the outer wall conforms to an inside surface of the aneurysm and the inner wall forms a first substantially tubular lumen to provide a path for blood flow. The system also includes a first expandable endoframe or endoframe disposed adjacent the filling structure. The first endoframe is radially expandable within at least a portion of the tubular lumen of the filling structure. In some embodiments, the system includes a second endograft or filling structure having a second endoframe disposed therein, each of the first and second filling structures positioned across the aneurysm. In such an embodiment, the first and filling structures are positioned side-by-side and parallel to one another in at least one plane.

The fenestrated nosecone is attached to the distal region of the inner shaft distal of the endograft. The nosecone has a through lumen, preferably a single through lumen, extending along a longitudinal axis of the nosecone. The nosecone is distally tapered to facilitate advancement of the inner shaft over a guidewire and includes a series of side ports in fluid communication with the through lumen for performing angiography. The system may include contrast media for performing angiography by injecting the contrast media through the guidewire lumen of the inner shaft, which then flows through the through lumen of the nosecone and out the side ports of the nosecone into the vasculature of the patient, preferably while the guidewire is disposed within the lumen. The guidewire lumen of the inner shaft and the lumen of the nosecone may be sized to allow for flow of a contrast media while the guidewire is disposed within the lumen. The through lumen of the nosecone may have a distal narrowed portion sized to slidably receive the guidewire so as to inhibit flow of the contrast media while the guidewire is disposed within, so that the contrast media is directed out through the series of side ports. The series of side ports are preferably pairs of side ports, each side port may be orthogonal to the through lumen. The series of side ports may be arranged in a helical pattern along the longitudinal axis of the nosecone, preferably distributed at regular intervals, such as every 90 degrees, along a helical path of the nosecone.

In many embodiments, the delivery system may include an outer sheath that is disposed at least partially over the filling structure and/or the endoframe. The system may also include a annular knob for retracting the outer sheath over a distal hypotube so as to expose the endograft for deployment at the aneurysm. The distal end of the outer sheath releasably engages with the nosecone at an interface creating a smooth transition between the outer surface of the nosecone and the outer surface of the outer sheath to facilitate advancement of the system through the vasculature. Preferably, the nosecone includes an isodiametric segment or a proximal region of the nosecone having the same outside diameter as the outer sheath, such that when interfaced the isodiametric segment is adjacent the distal end of the outer sheath having the same diameter, thereby creating a smooth transition. The isodiametric segment shifts deflection away from the outer sheath to nosecone interface, thereby maintaining a smooth transition. The interface may also include a radiopaque marker band, such as a gold marker band, on the nosecone and/or the outer sheath.

The system may further comprise an inflation device, such as a syringe, that is fluidly coupled with the filling structure and a pressure monitor. The pressure monitor may also be coupled with the filling structure so as to permit pressure monitoring of the filling structure as the filling structure is filled with the hardenable fluid filling medium. The pressure monitor may comprise a pressure gage, a digital or analog display or the like. The display may monitor a filling pressure in addition to other variables, including but not limited to elapsed time, filling time, and/or curing time. In one embodiment, the gauge includes electronics or a mechanism to filter out pressure spikes during filling. The gauge could include a relief valve to relieve pressure in the event of overfilling. In another embodiment, the pressure apparatus would calculate differential pressure between the inside of the filling structure and the patient's systolic/diastolic pressure in order to facilitate the filling medium process. In one embodiment, the digital display operatively couples near or on the handle so as to allow a physician to monitor pressure or other variables during the procedure. The digital pressure monitor may be battery-powered, detachable and disposable.

In many embodiments, the endoframe or endoframe is comprised of a metal and may be balloon expandable. The endoframe or filling structure may also carry a therapeutic agent that can be released therefrom in a controlled manner. Some therapeutic agents include anti-thrombogenics like heparin or agents which promote endothelial and smooth muscle cell growth, sealing and attachment. The filling structure may comprise a polymer.

The filling structure may also comprise a filling tube that is fluidly coupled therewith and that is adapted to fill the filling structure with the filling medium. The filling tube may also comprise an inner tube that is slidably disposed within an outer filling tube. Both the inner tube and the outer filling tube may be fluidly coupled with the filling structure. The method may comprise filling the filling structure with a fluid filling medium through the inner tube. The method may further comprise removing the inner tube and passing additional fluid filling medium through the filling tube after the inner tube has been removed.

In another aspect of the present invention, a system for treating an aneurysm in a blood vessel comprises an elongate flexible shaft having a proximal region and a distal region. An expandable member is disposed adjacent the distal region and a first expandable endoframe is disposed over the expandable member. The first endoframe is radially expandable from a collapsed configuration to an expanded configuration. A first double-walled filling structure is disposed over the first endoframe. The filling structure has an outer wall and an inner wall and the filling structure is adapted to be filled with a hardenable fluid filing medium so that the outer wall conforms to an inside surface of the aneurysm and the inner wall forms a first substantially tubular lumen to provide a path for blood flow. In the expanded configuration, the first endoframe engages the inner wall of the filling structure. A first releasable coupling mechanism releasably couples the filling structure with the flexible shaft and the coupling mechanism may comprise a tether that is releasably coupled with the filling structure and the flexible shaft. The coupling mechanism constrains axial movement of the filling structure relative to the flexible shaft.

The system includes coupling mechanisms for releasably coupling the filling structure and endoframe to the inner shaft. The coupling mechanisms may include a first and second releasable coupling mechanism. The first releasable coupling mechanism may comprise a first tether which couples the distal end of the endoframe with the inner shaft. In some embodiments, the proximal end of the filling structure may be coupled with the proximal region of the endoframe. In some embodiments, the first tether may comprise a tether loop with a release wire extending therethrough releasably coupling the endoframe to the inner shaft. Preferably, the first tether loop is fixedly attached to the inner shaft and withdrawn with the inner shaft after release of the filling structure and endoframe.

The endoframe may also be coupled with the filling structure. In an exemplary embodiment, the filling structure is coupled to a distal region of the endoframe with four sutures evenly distributed around its distal end. Each suture may be a single thread or strand between the filling structure and endoframe, or may include a suture loop. The suture attachment may include an eyelet coupling the suture loop to the endoframe. Thus, in many embodiments, the filling structure is coupled to the inner shaft through the attached endoframe.

The system may further comprise a second releasable coupling mechanism. The second mechanism may comprise a second tether or tether loop that is releasably coupled with the filling structure and the filling tube and/or inner shaft. The second tether may be attached to the filling structure at the opposite end of the filling structure as the first tether, so as to constrain axial movement of the filling structure relative to the flexible shaft. In many embodiments, the second tether comprises a tether loop attached to the filling tube, the loop extending through a suture on the filling structure and around the release wire, such that so long as the release wire remains within the tether loop the filling structure remains coupled to the filling tube.

The first and second tethers are further coupled to a release wire such that retracting the release wire, after deployment is complete, decouples the first and second tether from the filling structure so that the inner shaft can be removed from the deployed filling structure. The release wire is disposed adjacent the inner shaft and extends through the filling structure and over the endoframe. Once the filling structure and endoframe are deployed, the release wire can be retracted from inside a generally tubular lumen formed by the inner wall of the filling structure to release the filling structure from the inner shaft.

The method may further comprise pre-filling the first filling structure with a pre-filling fluid until the outer wall of the first filling structure conforms to the inside surface of the aneurysm, thereby unfurling the first filling structure. The pre-filling fluid may comprise saline and may be removed from the first filling structure. The method may also comprise pre-filling the first filling structure with pre-filling fluid until the outer wall of the first filling structure conforms to the inside surface of the aneurysm. The method may comprise performing angiography through the side ports of the fenestrated nosecone to detect potential endoleaks, and recording the pressure and filling volume of the first filling medium when endoleaks are no longer observed. The recorded pressure and volume of the pre-filling fluid used to pre-fill the first filling structure may be measured and then the pre-filling fluid may be removed from the first filling structure. Filling the first filling structure with the first fluid filling medium may comprise filling the first filling structure with the first filling medium using substantially the same pressure and volume as measured. The pre-filling fluid may comprise saline or contrast media to assist visualizing the filling process under x-ray fluoroscopy. The first filling medium may be passed through a filling tube that is fluidly coupled with the first filling structure.

Radially expanding the endoframe may comprise inflating a balloon that is disposed on the flexible shaft. Hardening the first fluid filling medium in the first filling structure may comprise polymerizing the first fluid filling medium in situ. In some embodiments, the first fluid filling medium may comprise polyethylene glycol.

A releasable coupling mechanism such as a tether may couple the first filling structure and/or endoframe with the flexible shaft and the step of releasing the first filling structure from the flexible shaft may comprise releasing the coupling mechanism or de-coupling the tether from the first filling structure or endoframe. One end of the tether may be releasably coupled with a release wire and the step of de-coupling the tether may comprise retracting the release wire thereby detaching the tether from the release wire. De-coupling the tether may comprise releasing the tether from a suture loop on the first filling structure. In some embodiments, a second releasable coupling mechanism, such as a tether may couple the first filling structure with the flexible shaft and the step of releasing the first filling structure from the flexible shaft may comprise de-coupling the second tether from the first filling structure. Releasing one or more of the coupling mechanisms may permit separation of a filling tube from the filling structure.

The method may further comprise the step of retracting an outer sheath away from the first filling structure and the first endoframe to allow expansion thereof. Pressure may be monitored during filling of the first filling structure. A filling tube may be released from the first filling structure after the hardenable filling medium has been delivered thereto. Releasing the filling tube may comprise detaching a filling tab coupled with the first filling structure.

In some embodiments, the method may further comprise providing a second elongate flexible shaft having a proximal end, a distal end, and a second expandable member near the distal end. The second flexible shaft may carry a second radially expandable endoframe over the second expandable member and a second double walled filling structure may be disposed over the second endoframe. The second shaft may be advanced in the vasculature of the patient so that the second filling structure is delivered to the aneurysm and the second filling structure is filled with a second fluid filling medium so that an outer wall of the second filling structure conforms to an inside surface of the aneurysm and an inner wall of the second filling structure forms a second substantially tubular lumen to provide a second blood flow path across the aneurysm. The second endoframe is radially expanded from a contracted configuration to an expanded configuration wherein in the expanded configuration the second endoframe engages the inner wall of the second filling structure. The second fluid filling medium may be hardened in the second filling structure and the second flexible shaft is released from the second filling structure. The second shaft may be retracted away from the second filling structure. Positioning, filling and release of the second filling structure generally takes the same form as described above with respect to the first filling structure.

The first filling structure may comprise a filling tube that is fluidly coupled therewith and the step of filling the first filling structure may comprise passing filling medium through the filling tube. The filling tube may comprise an inner tube that is slidably disposed therein and that is also fluidly coupled with the filling structure. The method may further comprise removing the inner tube from the filling tube and supplying additional filling medium to the filling structure by passing the filling medium through the filing tube after the inner tube has been removed therefrom.

These and other embodiments are described in further detail in the following description related to the appended drawing figures.

### BRIEF DESCRIPTION OF THE DRAWINGS

FIG. 1 illustrates the anatomy of an infrarenal abdominal aortic aneurysm.

FIG. 2 illustrates a delivery catheter shaft carrying a single prosthesis system which comprises a filling structure mounted over a endoframe structure.

FIG. 3 illustrates a system comprising a pair of prostheses for delivery to an infrarenal abdominal aortic aneurysm, where each prosthesis comprises a delivery catheter shaft carrying a filling structure mounted over a endoframe structure.

FIGS. 4A-4I illustrate exemplary usage of the system of FIG. 3 for treating an infrarenal abdominal aortic aneurysm.

FIGS. 5A-5B illustrates an exemplary system having a fenestrated nosecone during advancement of the system in the vasculature and during deployment of the filling structure at the treatment site, respectively.

FIG. 6 shows an exemplary aneurysm treatment system having a fenestrated nosecone, a carrier knob and a handle.

FIG. 7 illustrates the fenestrated nosecone of the system of FIG. 6.

FIG. 8 depicts angiography as performed with the nosecone of FIG. 6.

FIG. 9 illustrates the annular carrier knob of the system of FIG. 6.

FIG. 10 illustrates the handle of the system of FIG. 6.

FIG. 11 illustrates an exemplary pressure monitor in accordance with many embodiments.

FIGS. 12 and 13A-13C illustrate an exemplary embodiment and cross-sections of the embodiment.

FIGS. 14A-14E illustrates exemplary coupling mechanisms of an aneurysm treatment system, in accordance with several preferred embodiments.

### DETAILED DESCRIPTION OF THE INVENTION

The present invention relates generally to medical devices and medical methods, and more particularly to endoluminal systems for monitoring and delivering endoluminal treatments, such as endoframes, endografts, and other implantable structures. The systems may be used to monitor the body vessels and blood flow before, during and/or after treatment, and may be used to deploy endoluminal therapies, particularly therapies which involve treating aneurysms. The invention may include improved systems and methods for delivery and deployment of endoluminal therapies in conjunction with angiographic procedures. The invention may further include improved systems and methods for delivery of endografts, such as a filling structure, particularly filling structures having endoframes therein.

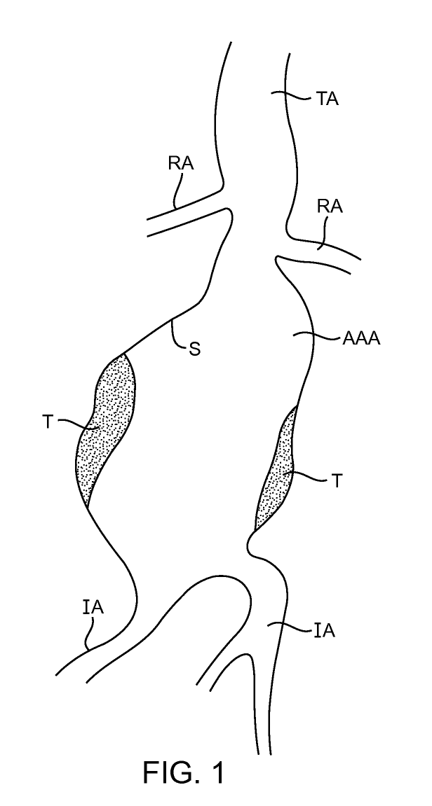

FIG. 1 illustrates the anatomy of an infrarenal abdominal aortic aneurysm comprising the thoracic aorta (TA) having renal arteries (RA) at an end above the iliac arteries (IA). The abdominal aortic aneurysm (AAA) typically forms between the renal arteries (RA) and the iliac arteries (IA) and may have regions of mural thrombus (T) over portions of its inner surface (S).

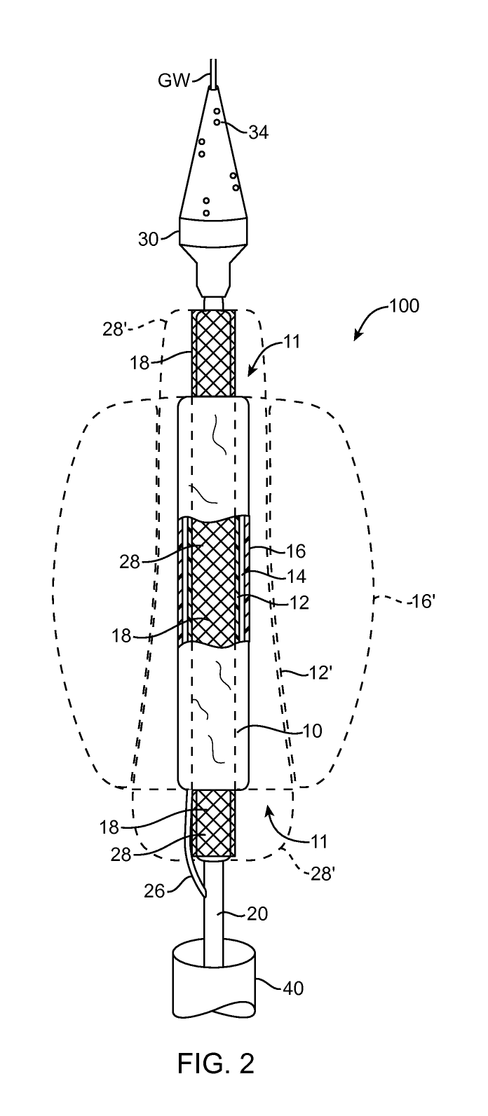

Referring now to FIG. 2, a system 100 constructed in accordance with the principles of the present invention for delivering a double-walled filling structure 10 (also referred to as an endograft in this disclosure) to an aneurysm includes the filling structure 10 disposed over a radially expandable endoframe 18, both of which are then mounted on a inner delivery catheter shaft 20 having an expandable element 28, typically an inflatable balloon, near its distal end and a nosecone 30 at its distal end. Nosecone 30 may be shaped to facilitate advancement of the delivery system through the vasculature and may include a series of side ports 34 for performing angiography before, during, or after deployment of the filling structure 10. An outer sheath 40 is slidably disposed over the inner delivery catheter shaft 20, the distal end of which interfaces with a proximal portion of nosecone 30 so as to facilitate advancement of the system through the vasculature of the patient. Expandable element 28 traverses the entire length of the endoframe 18 so that the endoframe 18 may be radially expanded upon expansion of the expandable element 28 (expanded balloon 28′ shown in broken line). Endoframe 18 traverses the entire length of filling structure 10 and most of endoframe 18 is covered by filling structure 10, however, endoframe 18 may also have proximal and a distal regions that extend uncovered beyond the filling structure 10. One of skill in the art will appreciate that lengths of the filling structure, endoframe and expandable element may be adjusted as required and thus the relative lengths are not limited to those disclosed above. Further details about the double-walled filling structure are disclosed in U.S. Patent Publication No. 2006/0212112 and preferred embodiments of an endoframe are disclosed in U.S. Patent Application Publication No. 2010/0004728, both of which the entire contents are incorporated herein by reference.

The catheter 20 will comprise a guidewire lumen (not shown), a balloon inflation lumen (not shown) or other structure for expanding other expandable components, and a filling tube 26 for delivering a filling medium or material to an internal space 14 of the double-walled filling structure 10. The internal space 14 is defined between an outer wall 16 and inner wall 12 of the filling structure. Upon inflation with the filling material or medium, the outer wall 16 will expand radially outwardly (expanded outer wall 16′ shown in broken line) as will the inner wall 12 (expanded inner wall 12′ shown in broken line). Expansion of the inner wall 12 defines an internal generally tubular lumen 11 through which blood flows after deployment of the filling structure in the aneurysm. The expandable balloon 28 or other structure will be expandable to correspondingly expand the endoframe 18 to provide support and to shape an inner surface of the lumen 11. In this embodiment, the expandable balloon is cylindrically shaped and therefore the generally tubular lumen 11 will also be cylindrically shaped. In other embodiments, the balloon may be pre-shaped to more precisely match the curvature of the vessel. For example, when treating an aortic aneurysm, a tapered, pre-shaped or curved balloon may be used so that the lumen substantially matches the aorta. Various balloon configurations may be used in order to match vessel tortuosity. Pre-shaped, curved or tapered balloons may be used in any of the embodiments disclosed herein in order to obtain a desired lumen shaped.

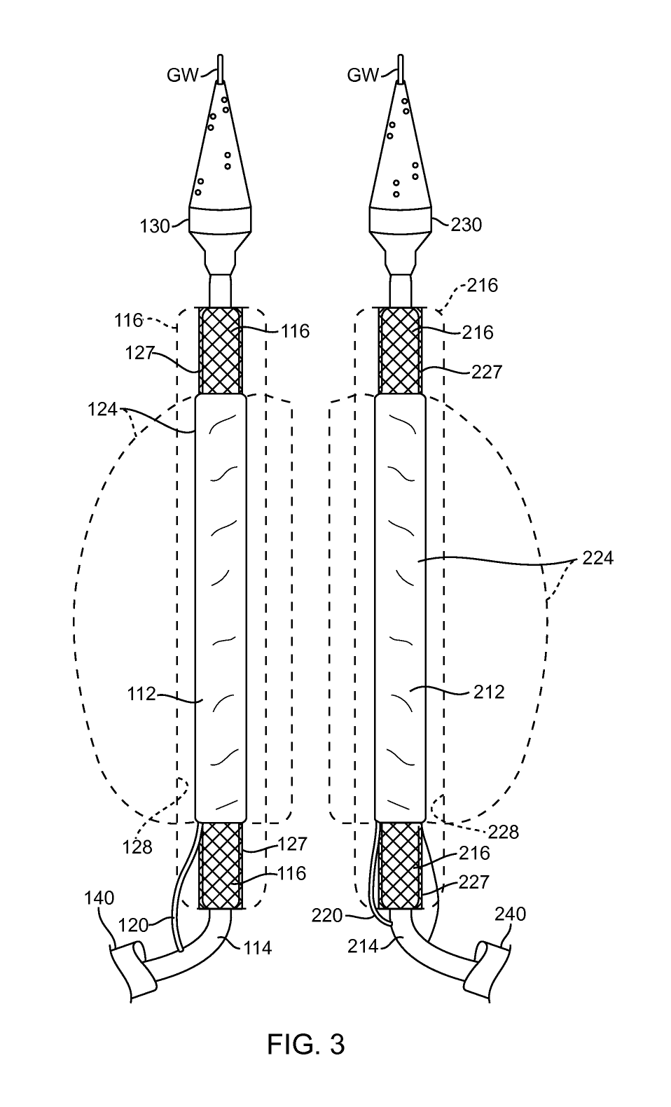

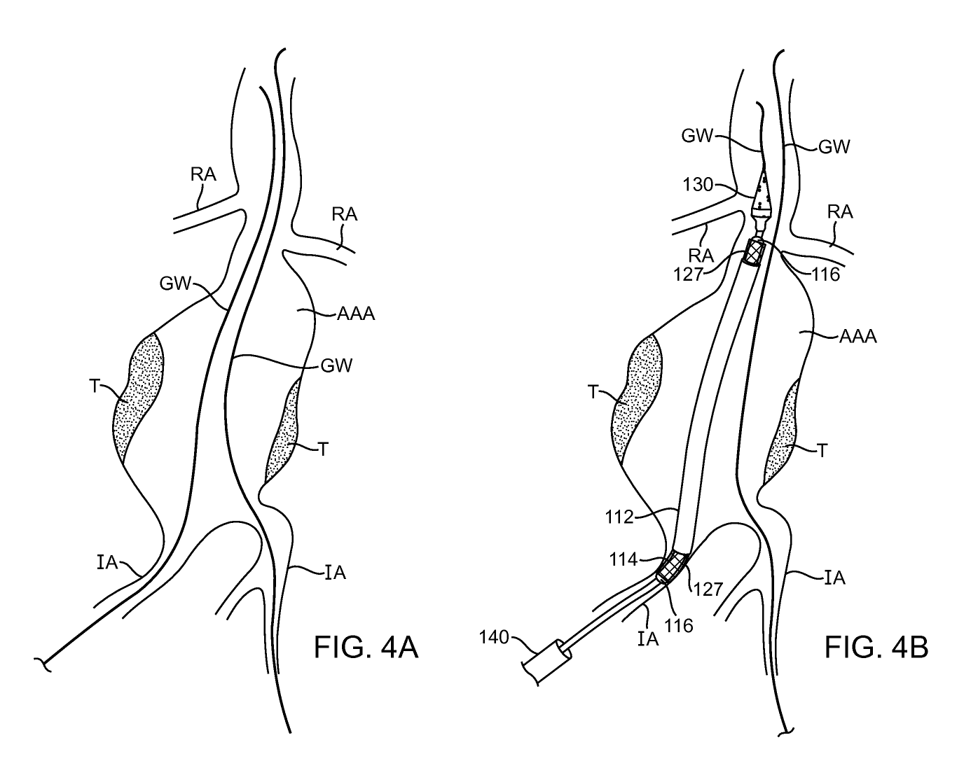

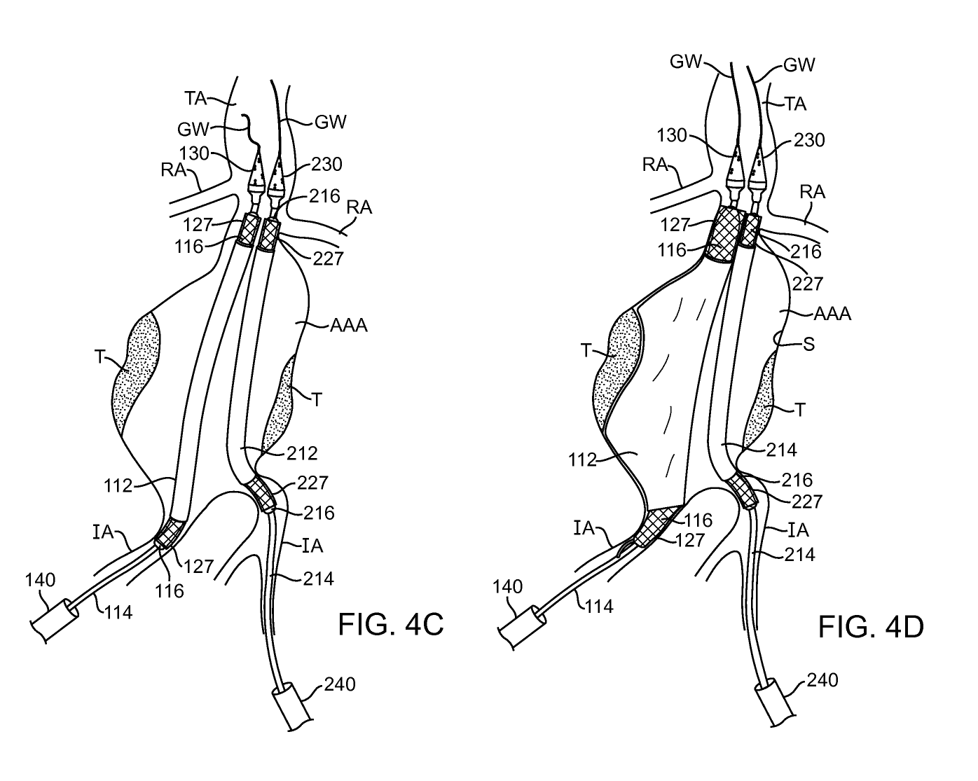

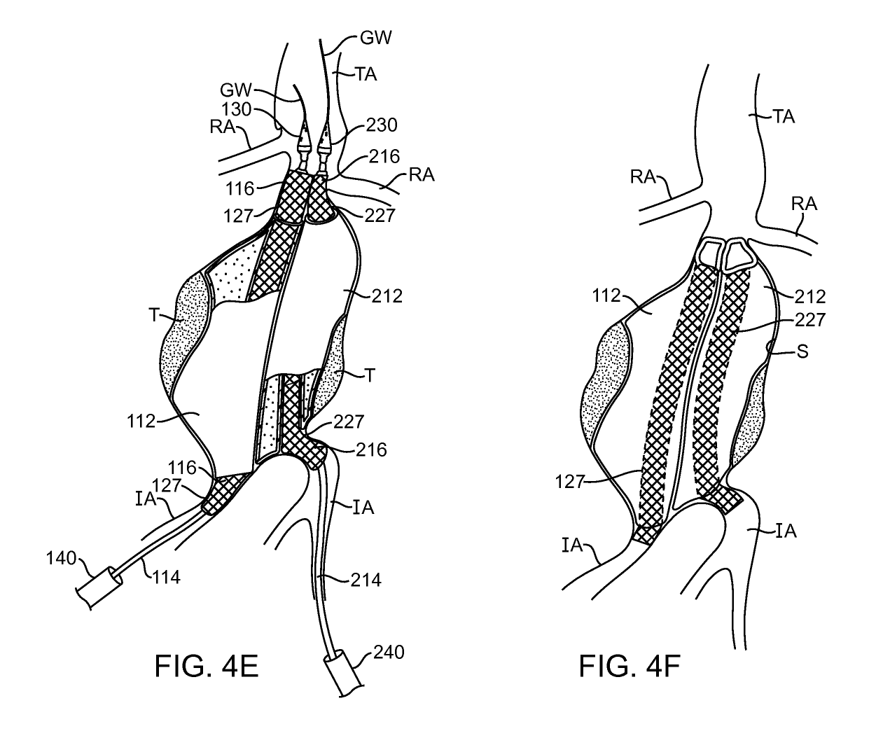

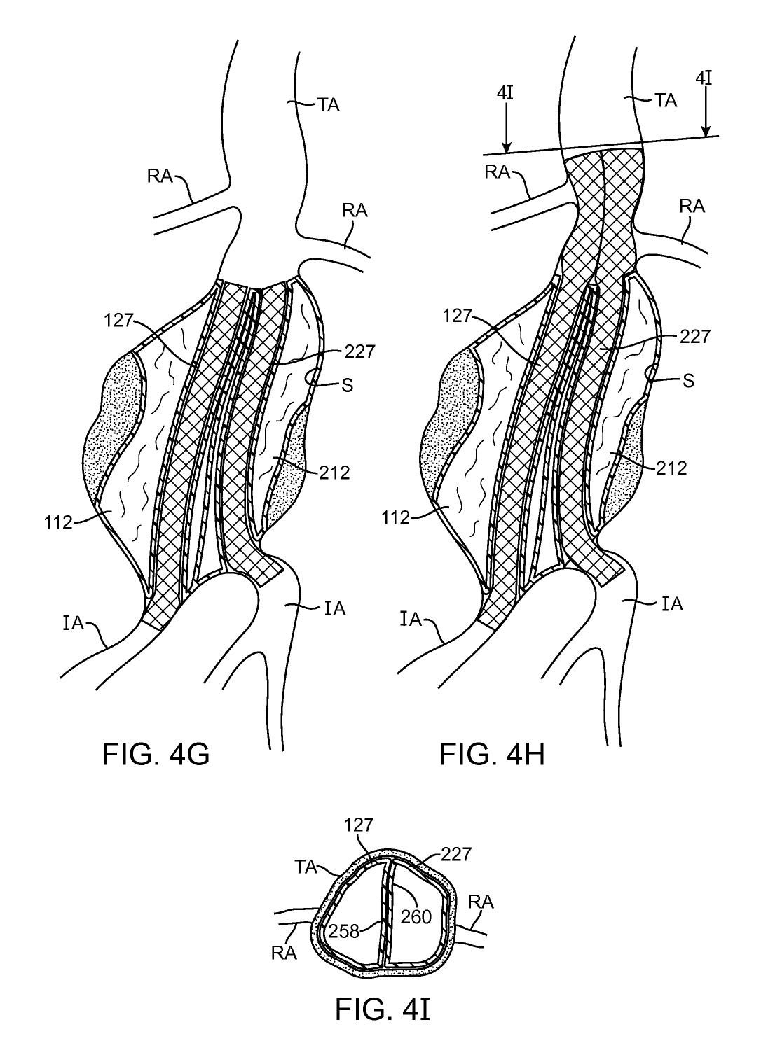

In a particular and preferred aspect of the present invention, a pair of double-walled filling structures will be used to treat infrarenal abdominal aortic aneurysms, instead of only a single filling structure as illustrated in FIG. 2. A system comprising such a pair of filling structures is illustrated in FIG. 3 which includes a first filling structure 112 and a second filling structure 212. Each of the filling structures 112 and 212 are mounted on delivery catheters 114 and 214, respectively and each system also has a radially expandable endoframe endoframe 127, 227. Inner delivery catheter shafts 114 and 214 include fenestrated nosecones 130, 230 for performing angiography before, during or after treatment, and outer sheaths 140, 240 slidably disposed over the filling structure during advancement of the system through the vasculature along the guidewires (GW). The components of the filling structures 112 and 212, the endoframes 127, 227 and inner delivery catheter shafts 114 and 214 are generally the same as those described previously with respect to the single filling structure system 10 of FIG. 2. Corresponding parts of each of the filling systems 112 and 212 will be given identical numbers with either the 100 base number or 200 base number. Filling structures 112 and 212 will generally be positioned adjacent each other within the aneurismal space to fill that space, as will be described with specific reference to FIGS. 4A-4I below.

FIGS. 4A-4I illustrate an exemplary use of the system in FIG. 3 for treating an infrarenal abdominal aortic aneurysm AAA with or without mural thrombus T. After the inner delivery catheter shafts 114 and 214 are advanced so that the filling structures 112 and 212 are positioned at the treatment site, the outer sheaths 140 and 240 are retracted relative the inner catheter shaft 114 and 214. The outer sheaths 140 and 240 are slidably disposed over the filling structures 112 and 212 and the corresponding endoframes 116 and 216 disposed therein. In FIG. 4A a pair of guidewires (GW) will first be introduced preferably percutaneously or by surgical cut down, from each of the iliac arteries (IA) and advanced across the aneurysm toward the renal arteries (RA). Referring now to FIG. 4B, the first delivery catheter 114 having a fenestrated nosecone 130 at its distal end and an expandable balloon 116 disposed on a distal portion will then be advanced over one of the guidewires GW to position the double-walled filling structure 112 across the aortic aneurysm (AAA) along with endoframe 127. An outer sheath 140, slidably disposed over the filling structure 112 and inner shaft 114, is retracted to expose the filling structure 112 at the target treatment site. The second inner catheter shaft 214 having expandable balloon 216 and fenestrated nosecone 230 at its distal end is then delivered over the other guidewire GW to position the second filling structure 212 adjacent to the first structure 112 across the aneurysm (AAA) along with endoframe 227, as illustrated in FIG. 4C. The outer sheath 240, slidably disposed over the filling structure 212 and inner shaft 214, is retracted to expose the second filling structure 212 at the target treatment site.

In an exemplary method, first, the balloon 116 or 216 is expanded. Expanding the balloon 116 or 216 expands the corresponding filling structure 112 or 212 and endoframe 127, 227 disposed thereon. Next, the expanded filling structure 112 or 212 is filled with the fluid filling medium. Then, the balloon 116 or 216 is deflated to allow a flow of blood through the filling structure 112, 212 filled with the fluid filling medium, while the expanded endoframe 127, 227 maintains the patency of the generally tubular lumen within the expanded filled filling structure 112 or 212. In alternative embodiments, one or both of filling structures 112 or 212 could be filled with the fluid filling medium first, then the balloon 116 or 216 expanded to expand the endoframe 127 or 227 to form a generally tubular lumen in the corresponding filling structure 112 or 212. In still another alternative embodiment, filling structure 112 or 212 could be filled with the fluid filling medium simultaneously with expanding the balloon 116 or 216 disposed therein. Typically, one of the filling structures 112, 212 and associated balloons 116, 216 will be expanded first along with the corresponding endoframe 127, 227, followed by the other filling structure, endoframe and balloon. In some embodiments, both balloons may be radially expanded simultaneously thereby also expanding the filling structures and endoframes simultaneously.

Alternatively, one or both filling structures 112, 212 may be filled with a hardenable material and then the filling structures 112, 212 are radially expanded along with the corresponding endoframe 127, 227. In still other embodiments, combinations of filling and expanding may be performed in different order depending on physician preference and aneurysm anatomy. In some embodiments, an optional pre-filling step may be performed prior to filling with the hardenable filling medium. In this optional step, once the delivery system is positioned across the aneurysm, the filling structure may be filled with CO₂ gas, contrast media, saline or other fluids to unfurl the filling structure 10 away from the delivery catheter thereby helping to ensure more uniform filling later on and reduce or eliminate striction between folds of the filling structure that may be present. During unfurling, the filling structure may be partially filled or fully filled so that it conforms to the inner aneurysm wall. Once unfurled, angiography may be performed through a fenestrated nosecone 130 or 230 on the inner catheters shafts 114 or 214 upstream of the aneurysm to detect leaks in the deployed filling structure 10. Once an optimal filling volume and pressure is determined to prevent the occurrence of endoleaks, the fluid may be removed from the filling structure and it may be filled with the hardenable material to expand and conform to the aneurismal space between the lumens and the inner aneurysm wall. Pressure relief valves such as those described herein may also be used to ensure that the filling structure is not over filled. In order to prevent overfilling of the filling structure, any of the pressure relief valves disclosed below may also be used to bleed off excess fluid from the filling structure.

FIG. 4D illustrates inflation of balloon 116 along with endoframe 127 in addition to expansion and filling of filling structure 112. The filling structure 112 and balloon 116 are expanded and inflated to fill generally half of the aneurismal volume, as illustrated in FIG. 4D. Filling and expansion can generally be carried out as described in U.S. Patent Publication No. 2006/0212112 for one filling structure, except of course that the filling structure 112 will be expanded to occupy only about one-half of the aneurismal volume. U.S. Patent Publication No. 2006/0212112 discloses filling of one filling structure in more detail including pressures, filling materials and other details, the entire contents of which have previously been incorporated herein by reference. After the first filling structure 112 has been filled, the second filling structure 212 may be filled and expanded along with endoframe 227, as illustrated in FIG. 4E. The second filling structure 212 may be deployed according to any of the exemplary methods described above with respect to deployment of the first filling structure. FIG. 4E also illustrates a cut away view of the expanded endoframes 127, 227 within the filled filling structures 112, 212. The upper ends of the balloons 116 and 216 will conform the tubular lumens of the filling structures against the walls of the aorta as well as against each other, while the lower ends of the balloons 116 and 216 will conform the tubular lumens into the respective iliac artery, IA. The expanded endoframe 127 not only provides support to filling structure 112, but also creates and shapes a lumen for blood passage from the aorta to one of the iliac arteries. Similarly, expanded endoframe 227 also provides a lumen for blood passage from the aorta into the other iliac artery. In some protocols filling of the filling structures (either both filled simultaneously or one after the other) may be performed before, during or after radial expansion of the balloons and the endoframe 127, 227 (either both expanded simultaneously or one after the other). Additionally, as discussed above with respect to FIG. 3, the endoframes 127, 227 may be radially expanded using a cylindrically shaped balloon to form a substantially cylindrically shaped lumen. Curved, tapered or pre-shaped balloons may also be used to expand the endoframes 127, 227, thereby forming a lumen that also is curved, tapered or shaped. The curved, tapered or pre-shaped balloon may be selected to match the anatomy of the vessel in which the endoframe and endograft is placed. Pre-shaped, curved or tapered balloons may be used in any of the other embodiments disclosed herein in order to obtain a desired lumen shape.

After filling the filling structures 112 and 212 as illustrated in FIG. 4E, the filling materials or medium will be cured or otherwise hardened as described in U.S. Patent Publication No. 2006/0212112 and the inner delivery catheter shafts 114 and 214 removed, respectively. The hardened filling structures along with the expanded endoframes 127, 227 will then provide a pair of tubular lumens opening from the aorta beneath the renal arteries to the right and left iliac arteries, as shown more clearly in broken line in FIG. 4F. The ability of the filling structures 112 and 212 to conform to the inner surface (S) of the aneurysm, as shown in FIG. 4F, helps the structures to remain immobilized within the aneurysm with little or no migration. Immobilization of the filling structures 112 and 212 may be further enhanced by providing any of the surface features described in U.S. Patent Publication No. 2006/0212112 which has been incorporated herein by reference.

The double filling structure embodiments preferably will include at least one endoframe deployed within each of the tubular blood flow lumens. The endoframes will generally be endoskeletal structures that lay the foundation for new lumens, and will be deployed within the tubular lumens of the double-walled filling structures using balloon or other expansion catheters (in the case of malleable or balloon-expandable endoframes) and an optional retractable constraining sheath. FIG. 4G more clearly shows the first endoframe 127 disposed within the generally tubular lumen of the first filling structure 112 while a second endoframe 227 is disposed in the tubular lumen of the second filling structure 212. As illustrated, in this exemplary embodiment, the endoframes are balloon expandable structures which extend into the iliac arteries IA at the lower end of the filling structures. In other embodiments, the endoframes may be self-expanding endoframe-like structures fabricated from a shape memory alloy such as Nitinol.

Referring now to FIG. 4H, first and second endoframes 127 and 227 may extend upwardly on the aortic side of the first and second filling structures 112 and 212. In such embodiments, the first and second endoframes 127 and 227 may be constructed so as to not obstruct the renal arteries. For example, the first and second filling structure 112 and 212 may include a side hole so as to allow flow of blood through the renal arteries (RA). When the endoframe structures extend into the thoracic aorta TA, it will usually be desirable that they be expanded so that they conform to each other along a plane or region of contact. For example, as shown in FIG. 4I, the upper ends of the endoframes 127, 227 may be formed preferentially to have D-shaped cross-sections when expanded, although other cross-sections such as elliptical, circular, etc. may be formed. Thus, flat faces 258 and 260 will engage each other with the remaining portion of the endoframe conforming to the inner wall of the aorta. In this way, most of the cross-sectional area of the aorta will be covered with the endoframe, thus enhancing blood flow through the filling structures. Other configurations are disclosed in U.S. Patent Publication No. 2006/0212112 previously incorporated herein by reference.

In the exemplary embodiment of FIGS. 4A-4I, each endoframe and filling structure is both disposed coaxially and generally concentrically over an expandable member coupled to a delivery catheter and the entire system is delivered to the aneurysm at one time. Such a coaxial and concentric system, such as that shown in FIG. 2, typically includes a filling structure 10, also referred to as an endograft that is coaxially disposed over the endoframe 18, both of which are then coaxially and concentrically positioned over a radially expandable balloon 28 which is coupled to the distal region of a inner shaft 20. Proximal and distal portions of endoframe 18 may extend uncovered by filling structure 10 and a filling tube 26 allows a fluid to be delivered to the filling structure 10. While this embodiment is promising, under certain conditions, endoleaks occur, thereby resulting in incomplete occlusion of the aneurysm. Although a separate angiography catheter may be useful to visualize the blood flow through the aneurysm and detect endoleaks during deployment, a separate angiography catheter would likely increase the profile and further complicate the method of delivery. Additionally, in certain situations, the filling structure may move relative to the endoframe during delivery, thereby resulting in inaccurate placement of one or both devices. It would therefore be advantageous an integrated system provide a system that can perform an angiography procedure without substantially increasing the profile of the delivery system or over complicating the procedure. It would be further advantageous for such a system to provide more effective ways of coupling the filling structure and endoframe to the delivery catheter to inhibit movement and facilitate more accurate delivery of the endoframe and endograft to the treatment site.

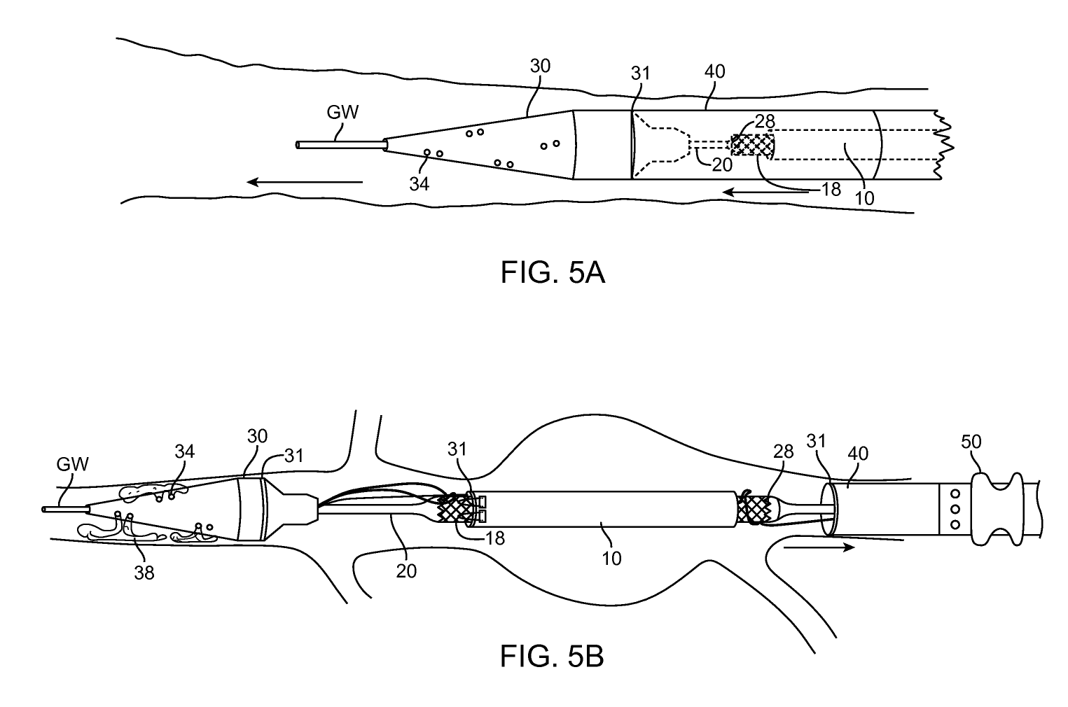

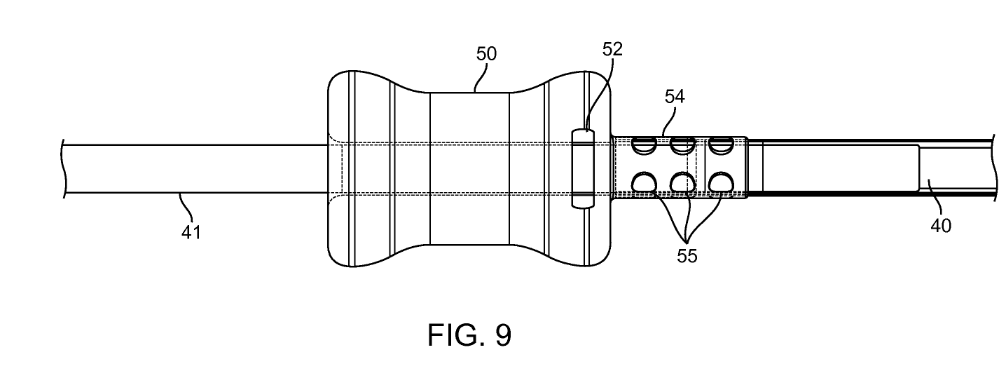

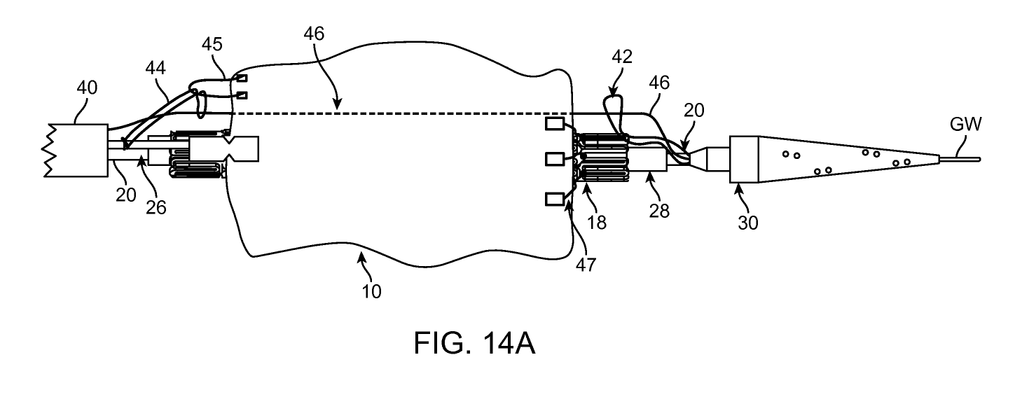

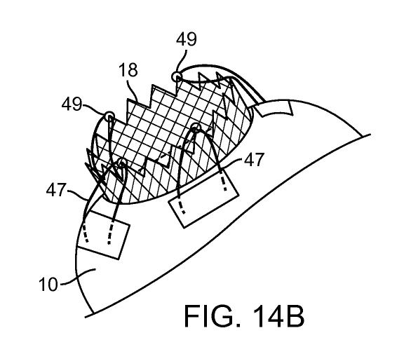

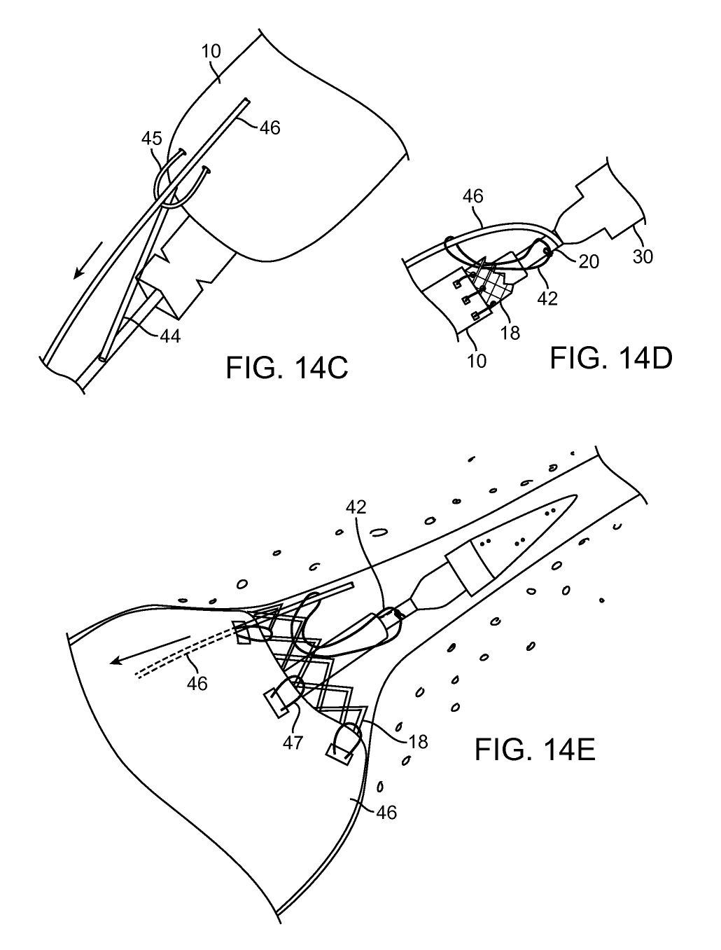

FIGS. 5A-5B illustrate an exemplary embodiment of a delivery system having a fenestrated nosecone 30 and a retractable outer sheath 40, as shown both during delivery of the system to the aneurysm (FIG. 5A) and during deployment of the filling structure at the aneurysm (FIG. 5B). The exemplary embodiment includes an inner delivery catheter 20 having a filling structure 10 and endoframe 18 disposed thereon, and an outer sheath 40 slidably disposed over the inner shaft 20, filling structure 10 and endoframe 18. Nosecone 30, attached to the distal end of inner shaft 20, includes a lumen extending therethrough, preferably a single through lumen, the through lumen connected to a guidewire lumen of the inner shaft 20, such that the nosecone and inner shaft can be simultaneously advanced along the same guidewire GW. The nosecone includes a series of sideports 34 in fluid communication with the through lumen for performing angiography before, during or after deployment of the filling structure 10. The system includes releasable coupling mechanisms to attach filling 10 and endoframe 18 to inner delivery catheter 20 during delivery of the system to the aneurysm (shown in detail in FIGS. 14A-14E). The distal end of outer sheath 40 releasably couples with the nosecone to facilitate advancement of the system through a patient's vasculature, as illustrated in FIG. 5A. A marker band 31 may be disposed near the interface on one or both of the proximal portion of nosecone 30 and the distal end of the outer sheath 40, so as to allow a user to visualize the location of the outer sheath 40 relative to the filling structure 10. Once the filling structure is positioned as desired, outer sheath 40 can be retracted relative to the filling structure 10 and inner shaft 20, as shown in FIG. 5B, thereby exposing the filling structure for deployment within the aneurysm. The fenestrated nosecone remains attached to the end of the inner shaft 20 distal of the filling structure 10 so that an angiography procedure may be performed. For example, as shown in FIG. 5B, a contrast media 58 may be injected through the guidewire lumen of the inner shaft 20 and out through the side ports 34 of the nosecone 30 into the vasculature. In a preferred embodiment, outer sheath 40 is retracted by pulling proximally on an annular knob 50 depicted in FIG. 9. After deployment and release of the filling structure and endoframe from the inner catheter, outer sheath 40 can be advanced, typically using knob 50, so as to interface with nosecone 30 to facilitate withdrawal of the system from the patient.

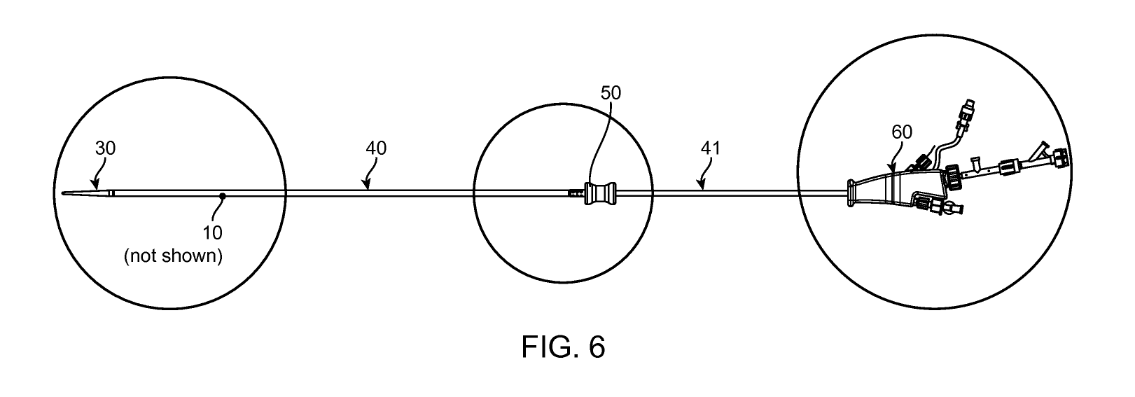

FIG. 6 shows an exemplary embodiment of the delivery system, which includes the fenestrated nosecone 30, an outer sheath 40, a filling structure 10 (not shown) disposed within outer sheath 40, an annular carrier knob 50 for retracting the outer sheath 40, and a handle 60. In an exemplary method of treating an aneurysm, the system, as shown in FIG. 6, is inserted into the vasculature of the patient. The distal tapered shape of nosecone 30 and the smooth transition between the nosecone 30 and outer sheath 40 coupled thereto facilitates advancement of the system through the vasculature of the patient. Once the filling structure 10 is positioned at the aneurysm treatment site, the physician may retract the outer sheath 40 by pulling carrier knob 50 proximally, thereby retracting outer sheath 40 over hypotube 41 proximal of knob 50. The carrier knob 50 is fixedly attached to the proximal end of outer sheath 40 and includes a seal between outer sheath 40 and hypotube 41. Handle 60, disposed at the proximal end of inner shaft 20 is used to advance and withdraw the system, as well as to deploy the fillable structure 10 or perform angiography through nosecone 30.

Fenestrated Nosecone

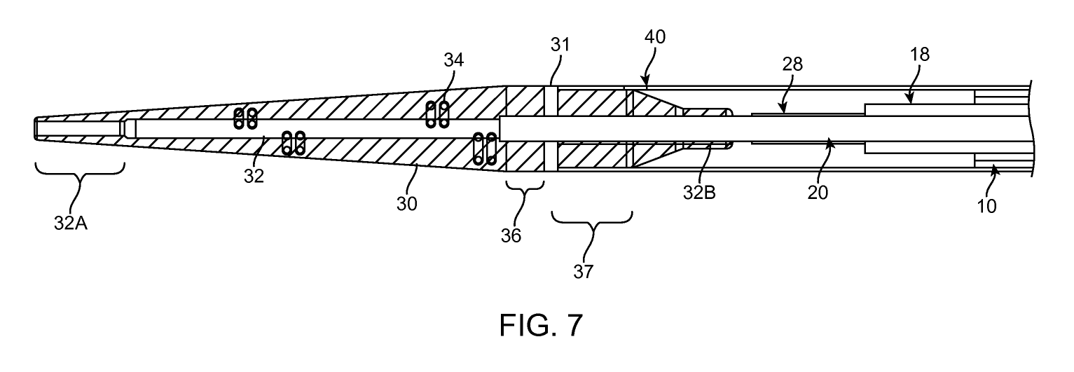

FIG. 7 illustrates the nosecone 30 of the embodiment of FIG. 6 in more detail. Although shown in the embodiment of FIG. 6, nosecone 30 may be incorporated into any of the embodiments disclosed herein. Nosecone 30 is distally tapered and includes a through lumen 32 extending along a longitudinal axis of the nosecone to facilitate advancement of the system over a guidewire through the vasculature. Nosecone 30 is also fenestrated having a series of sideports in fluid communication with the through lumen for performing angiography. The through lumen 32 of the nosecone is sized so as to simultaneously receive a guidewire and a flow of contrast media for performing angiography through the side ports 34. The fenestrated nosecone 30 is advantageous as it facilitates both advancement of the system over a guidewire and delivery of contrast media in an angiography procedure through the same lumen while the guidewire is disposed therein, thereby simplifying the procedure while maintaining a reduce profile.

To facilitate both advancement of the system and deployment of the filling structure, fenestrated nosecone 30 releasably couples or interfaces with the distal end of outer sheath 40. In an exemplary embodiment, nosecone 30 includes an isodiametric portion 36 which is isodiametric with the outer diameter of the distal end of outer sheath 40 so as to create a smooth transition at the interface and prevent “snowplowing” against a vessel wall as the system advances through the patient's vasculature. Isodiametric portion 36 extends a distance distal of the interface so as to increase the stiffness of the nosecone near the interface and prevent flexure of the nosecone and/or outer sheath at the interface during advancement of the system. Nosecone 30 may also include a portion 37 having an outside diameter slightly smaller than the inside diameter of the distal end of outer sheath 40; thus, portion 37 is disposed within the outer sheath 40 such that the outer sheath 40 fittingly receives portion 37 so as to releasably couple with the nosecone 30. Portion 37 may extend a distance proximal of the interface so as to increase the stability of the coupling and to increase the stiffness near the interface to prevent separation between the nosecone and outer sheath 40 as the system winds through complex or tortuous vasculature, thereby further reinforcing the smooth transition between the nosecone 30 and the outer sheath 40 to reduce the likelihood of “snowplowing” against the vessel wall. The nosecone 30 and/or the outer sheath 40 may further include a radiopaque marker band 31 near the interface to allow a physician to image the location of the distal end of the outer sheath 40 relative to nosecone 30. The resin to manufacture the nosecone may also include a radiopaque filler to facilitate imaging during the procedure.

Nosecone 30 is dimensioned to facilitate advancement in a patient's vasculature. An exemplary nosecone may be between 1 to 4 inches in length, preferably 2.5 to 3 inches, and have an outside diameter tapering from about 0.25 inches at a proximal end to about 0.050 inches at a distal end. The through lumen 32 extending through the nosecone 30 may also reduce in size as the outside diameter tapers down. For example, the through lumen at the proximal end 32B may be between 0.05 and 0.1 inches, preferably about 0.08 inches, and reduce in size gradually or incrementally to 0.02 to 0.04 inches, preferably about 0.05 inches at the distal portion 32A of nosecone 30.

Angiography may be performed by injecting a contrast media through the guidewire lumen of the inner shaft 20, which then flow into the through lumen 32 of nosecone 30 and out the side ports 34, preferably while the guidewire GW is disposed within the guidewire lumen and through lumen 32. Angiography may be performed through nosecone 30 before, during, or after treatment of the aneurysm, and is useful for imaging the flow of blood, particularly for detecting leaks in an endograft deployed in an aneurysm. In an angiography procedure, a radiopaque contrast media is delivered into a blood vessel and an X-ray based imaging technique, such as fluoroscopy, is used to image the flow of the contrast media as it flows through the blood vessel. Preferably, the contrast media is released uniformly into the blood vessel so that the imaging produces a more accurate representation of blood flow through the vessel.

In an exemplary embodiment, fenestrated nosecone 30 includes pairs of side ports 34 arranged in a series equally distributed along the nosecone in a helical fashion so as to evenly distribute the contrast media into the vasculature. Preferably, each of side ports 34 extends in an orthogonal direction from the longitudinal axis of the nosecone 30. In an exemplary embodiment, the nosecone 30 comprises a series of 4 to 10 pairs of side ports 34, preferably 5 to 7 pairs of side ports, arranged in a helical fashion as described above. Typically, a side port 34 has a diameter within a range of 0.01 inches to 0.05 inches, and more preferably within a range of 0.03 to 0.04 inches.

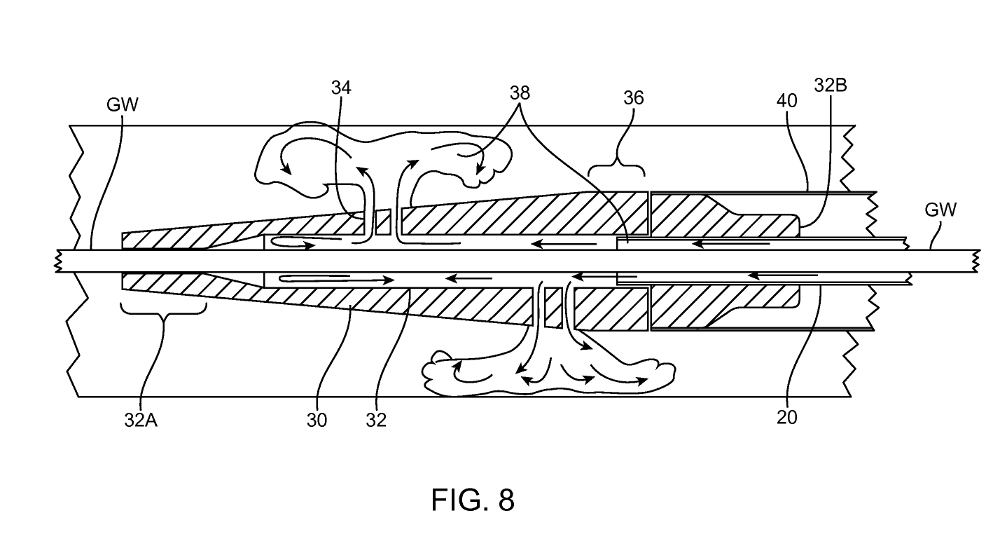

FIG. 8 illustrates the nosecone 30 having a guidewire GW disposed within guidewire lumen 32 as well as a flow of contrast media 38 through a portion of guidewire lumen 32. In an exemplary embodiment, the guidewire lumen 32 includes a distal portion 32A which is reduced in size from the guidewire lumen 32 at the proximal end of nosecone 30. Distal portion 32A of the through lumen 32 is sized to slidably receive the guidewire and fit so as to inhibit flow of contrast media through the distal opening when the guidewire is disposed therein. The portion of the through lumen 32 proximal of portion 32A, however, is sized to simultaneously receive the guidewire and facilitate flow of contrast media 38. The narrowed distal region 32A of through lumen 32 substantially inhibits flow of contrast media through the distal opening of the nosecone, thereby directing the flow of contrast media through the side ports 34 and into the vasculature of the patient, as illustrated in FIG. 8.

Annular Knob

FIG. 9 illustrates the carrier knob of the system of FIG. 6, the carrier knob coupling the proximal end of outer sheath 40 with hypotube 41. In such an embodiment, the outer sheath 40 is slidably disposed, at least partially, over hypotube 41 or main catheter shaft when outer sheath 40 is retracted. In an exemplary method, outer sheath 40 is retracted relative to the inner shaft 20 to expose the filling structure 10, endoframe 18 and expandable member 28 on a distal region of inner shaft 20. The carrier knob 50 is fixedly coupled to the proximal end of the outer sheath 40, preferably with a heat bond, so that a physician may manually pull the carrier knob 50 to retract outer sheath 40 over hypotube 41. Carrier knob 50 may also include an internal seal 52 and a strain relief element 54 heat bonded to the proximal end of outer sheath 40. The carrier knob 50 may comprise any material, such as polyetheramide, which can be bonded with the outer sheath 40.

Carrier knob 50 may include an internal seal 52, such as an silicone O-ring, to prevent fluid outflow from between the distal and proximal segments of the outer sheath 40. Typically, the blood pressure of the patient against internal seal 52 is sufficient to prevent fluid outflow from the body between outer sheath 40 and hypotube 41. As the carrier knob 50 is pulled proximally internal seal 52 slides proximally contacting the outer surface of the hypotube 41 and inside the annular knob 50.

Strain relief element 54 may comprise a tubular element fixedly attached to the distal region of carrier knob 50 and is sized to circumscribe the proximal end of outer sheath 40. The tubular strain relief element 54 may include a plurality of holes 55 which absorb stress and strain from the outer sheath 40 to prevent breakage of the heat bond. Carrier knob 50 may be heat bonded to the outer sheath 40 by covering the proximal end of outer sheath 40 by a laminate material, preferably a polymer material such as Pebax®, placing the tubular element 54 over the laminate covered outer sheath 40, and heating the assembly so that the laminate bonds to the outer shaft 40 and/or the knob 50, preferably bonding with both the outside surface of outer sheath 40 and the inside surface of the tubular strain relief element 54. In an exemplary embodiment, the laminate flows through the holes 55 in the tubular element 54 when heated strengthening the bond between the outer shaft 40 and knob 50. In some embodiments, additional layers of laminate may be placed over the strain relief member 54 during the heat bonding process. The hypotube 41 or main catheter shaft is inserted through the knob 50 from the side opposite the tubular strain relief member and through the proximal end of the outer sheath 40. The system may further include a retaining mechanism to prevent the hypotube 41 from slipping out of the knob 50 when the outer sheath 40 is advanced. The retaining mechanism may comprise a collar, a ring, a pin, or any mechanism suitable to maintain a slidably coupling between the hypotube 41 and the annular knob 50. The system may also include a keying mechanism to prevent rotation between hypotube 41 (main catheter shaft) and sheath 40.

Handle

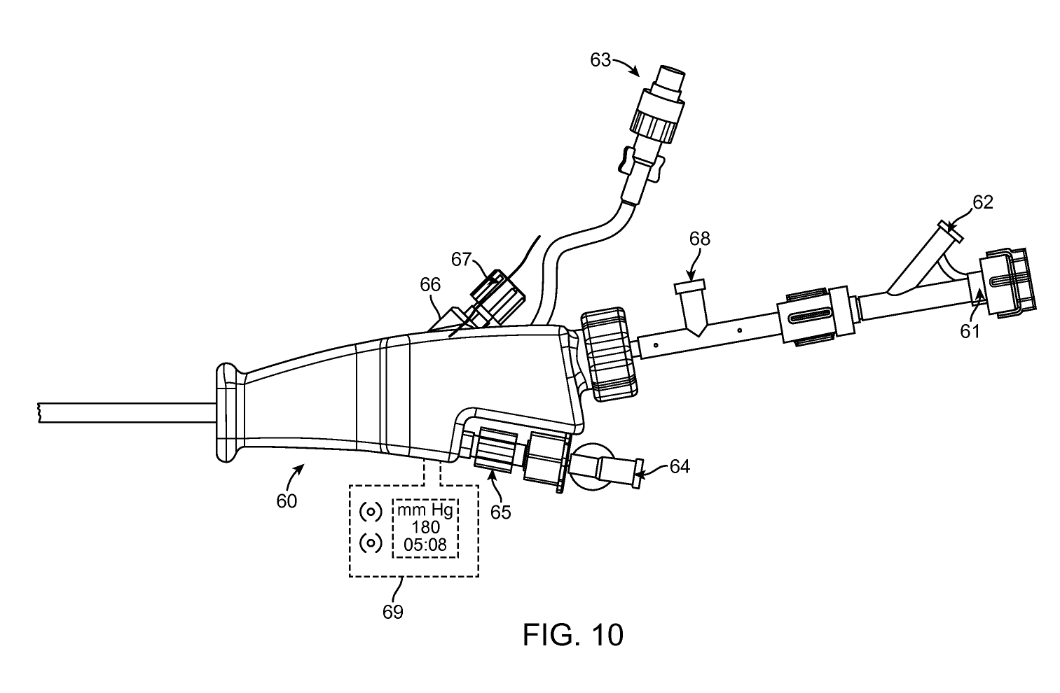

FIG. 10 illustrates a handle 60 of an exemplary delivery system of FIG. 6. Handle 60 include a body having a guidewire access port 61, an angiography port 62, a flush line 63, an inner and outer fill tube port 64, 65, a release wire port 66, and a balloon inflation port 68. The angiography port 62 provides access to the guidewire lumen such that contrast media can be injected through the guidewire lumen and out the series of side ports 34 of the nosecone 30. As the contrast media may be viscous, the backend pressure needed to perform angiography may reach 1,000 psi or more. These pressures may potentially cause the guidewire to “shoot” out of the guidewire lumen. To prevent this from happening, the guidewire access port 61 may include a locking mechanism for locking guidewire GW to the handle during the angiography process. Thus, angiography port 62 provides access to the guidewire lumen, while the guidewire access 61 may seal and lock the guidewire GW into place. Release wire access port 66 provides access for removal of the release wire so as to decouple the coupling mechanisms and release the deployed filling structure from the inner shaft 20. Release wire access port 66 may include a luer cap 67, which may be attached to the proximal end of the release wire, such that removing the cap and pulling in the proximal direction will retract the release wire and decouple the coupling mechanisms. Optionally, handle 60 may rotate on inner and outer shafts to prevent undesired transfer of torque on the catheter shafts. The handle 60 may also include a pressure monitor display 69 so that a physician may monitor the filling pressure during the treatment procedure. Alternatively, the pressure monitoring system and/or display may be a separate component from the handle. Typically, the pressure monitor is fluidly coupled with the inner and/or outer filling tube so as to monitor the filling pressure during filling of the filling structure 10 with the hardenable medium.

Pressure Monitoring

In an exemplary method of deploying a filling structure and endoframe, pressure monitoring may be utilized in the following way. After two filling structures have been delivered to the treatment site, both endoframes are radially expanded to help create a lumen for blood flow through the filling structure across the aneurysm. Using data from a patient's computerized tomography (CT) scans, a fill volume of the aneurysm treatment site may be estimated and then divided by two, half for each of the two filling structures. This represents the baseline filling volume for each filling structure and is the minimum volume of filling material to be injected into each of the filling structures. Syringes or other injection devices coupled with a pressure gage may be used to optionally pre-fill each filling structure with contrast material using the baseline volume and the resulting baseline fill pressure may be noted. In addition, the pressure monitoring system may include a pop-off valve to relieve excess pressure in the event of overfilling. The pressure monitoring system may include a sensor inside the filling structure and a sensor monitoring the patient's systolic and or diastolic blood pressure used for calculating the patient's differential blood pressure. A differential blood pressure reading between the filling structure and the patient's systolic and or diastolic pressure simplifies the process of filling the structures as this allows the physician to accurately target the pressure inside the filling structure. This allows unfurling of the filling structure and provides a preliminary assessment of how the expanded filling structures fit into the aneurismal space. Once this is accomplished, the contrast material is removed from the filling structures (in a method utilizing a pre-filling step). Again using the patient CT data, a functional fill volume may be determined. This volume is a percentage of the aneurysm volume obtained from the CT data, or it may be a predetermined number and is the volume of filling material that effectively seals and excludes the aneurysm. Functional fill pressure will be the pressure at which the functional fill volume is attained. A polymer fill dispenser may then be used to fill each filling structure with the functional fill volume and the functional fill pressure is noted. While holding the functional fill volume and pressure, the filling structure may be observed under fluoroscopy to check for proper positioning, filling and the absence of leakage across the aneurysm. If leaks are observed, additional polymer may be added to the filling structures until the leaks are prevented or minimized. Excessive additional polymer should not be added to the filling structure in order to avoid exceeding a safe fill volume or safe fill pressure. Once the physician is satisfied with the filling and positioning of the filling structures, stopcocks to the filling structures may be closed to allow the polymer to harden and then the delivery devices may be removed from the patient.

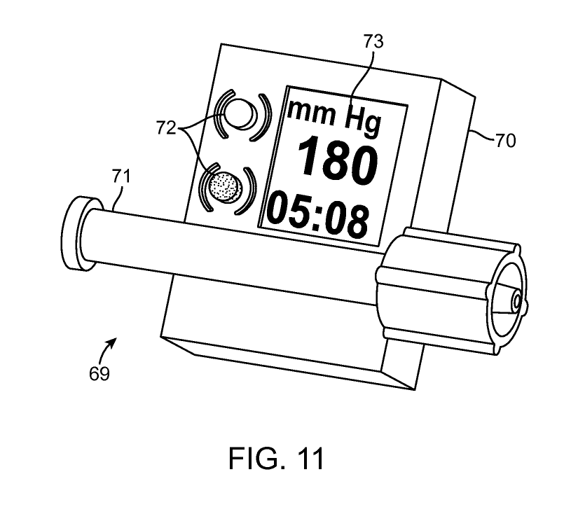

FIG. 11 illustrates a pressure monitor 69 comprising an integrated pressure gauge transducer. Pressure monitor 69 may include a body 70, a pressure fitting 71, control buttons 72, and a digital display 73 for reading the pressure and time output. The body 70 may include pressure transducers for determining the pressure and/or differential pressure. Pressure fitting 71 couples the pressure transducers of the body 70 to the filling system, control buttons 72 allow a user to adjust the pressure or the pressure or time readout. The pressure fitting 71 may include a tubular member and a luer cap for fluidly coupling with a filling line of the delivery system 100. Pressure monitor 69, or a portion thereof, may be re-usable or disposable, preferably the pressure transducers are for a single use and are disposable. Optionally, the pressure monitor 69 may be incorporated into the handle 50, as discussed above. In addition to actual pressure monitoring by gages and graphical displays, etc., other pressure indicators may also be used to facilitate determining the filling status of the filling structure. For example, some embodiments may employ a relief valve. The relief valve is preset to a certain pressure such that beyond the preset pressure, any additional filling material will bleed out of the filling structure. While the relief valve may be adjacent the filling structure, preferably the filling material will be vented toward the proximal end (handle end) of the catheter, outside the body. This keeps potentially dangerous fluids or other filling material from being introduced into the body.

The methods may also include use of a pressure monitoring system that allows a physician to monitor the pressure before, during or after filling the filling structure. The system may include a user interface for displaying the pressure output and may optionally include a time output displayed in conjunction with the pressure output. The monitoring system may also include a processor for analyzing the pressure data. The processor may be programmed to normalize the pressure data. The pressure monitoring system enables the physician to closely monitor the filling pressure during deployment of the filling structure and may include additional functions to facilitate proper deployment, including: pressure cut-off switches, warning indicators if the measured filling pressure is outside an acceptable range, and pressure bleed off switches to optimize filling pressure and compensate for fluctuations in pressure during filling. The monitoring system may also record the pressure data during deployment. Additionally, in embodiments deploying two filling structures, the monitoring systems may be operatively coupled such that the system or a physician may monitor pressures during filling of one filling structure in response to pressure output readings from the other adjacent filling structures.

Pressure monitoring can also be performed at various stages of the aneurysm repair procedure to help control the filling process of the filling structure. The monitoring of pressures serves to reduce the risk of dissection, rupture or damage to the aneurysm from over-pressurization and also can be used to determine an endpoint for filling. Monitoring can be done before, during or after filling and hardening of the filling structure with filling medium. Specific pressures which can be monitored include the pressure within the internal space of the filling structure as well as the pressure in the space between the external walls of the filling structure and the inner wall of the aneurysm. A composite measurement can also be made combining pressures such as those measured within the interior space of the filling structure, together with that in the space between the external walls of the structure and the aneurysm wall or other space at the aneurysm site and an external delivery pressure used by a fluid delivery device, such as a pump or syringe, to deliver the filling medium. Control decisions can be made using any one of these pressure measurements or a combination thereof. Methods of pressure monitoring are discussed in detail in related commonly-owned applications: U.S. patent application Ser. No. 11/482,503 and U.S. patent application Ser. No. 12/429,474, the entire contents of which are incorporated herein by reference.

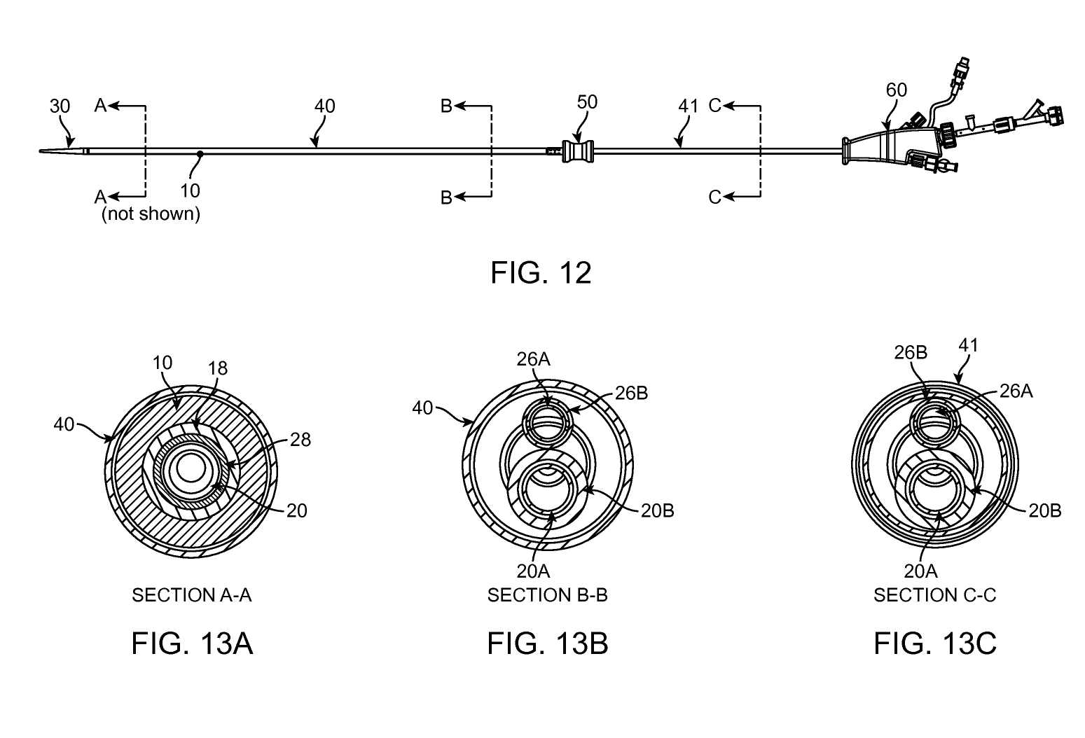

FIG. 12 illustrates and exemplary delivery system and FIGS. 13A-13C illustrate cross-sections of the delivery system of FIG. 12. FIG. 13A shows cross-section A-A at a location proximal of the nosecone at which the filling structure 10, endoframe 18 and balloon 28 are each disposed concentrically outside inner shaft 20 and inside of outer sheath 40. FIG. 13B shows cross-section B-B at a location distal of the carrier knob 50 at which concentric inner and outer filling tubes 26A, 26B are disposed adjacent concentric inner and outer balloon shafts 20A, 20B of inner shaft 20 within outer sheath 40. FIG. 13C shows cross-section C-C at a location between the handle 60 and carrier knob 50 at which concentric inner and outer filling tubes 26A, 26B are disposed adjacent concentric inner and outer balloon shafts 20A, 20B of inner shaft 20 within hypotube 41. In many embodiments, the outer sheath 40 and hypotube 41 comprise different materials. For example, the distal segment of outer sheath 40 may comprise polyether block amide, while the segment of outer sheath 40 proximal of the carrier knob 50 may comprise 304 stainless steel.

Delivery System Coupling Mechanisms

FIGS. 14A-14E illustrates coupling mechanisms of an exemplary delivery system. The coupling mechanisms inhibit movement of the filling structure and endoframe relative to the delivery catheter so as to facilitate positioning of the filling structure and endoframe at the aneurysm. FIG. 14A depicts the coupling mechanisms during deployment of the filling structure, wherein the outer sheath 40 has been retracted and filling structure 10 has been partially filled. The coupling mechanisms may include tethers 42, 44 and/or tether loops for attaching filling structure 10 and endoframe 18 to delivery catheter 20. As shown in FIG. 14A, filling structure 10 is disposed over endoframe 18 which is crimped onto expandable member 28 disposed near the distal end of inner shaft 20. Two releasable coupling mechanisms, tethers 42 and 44, along with release wire 46 inhibit movement of filling structure 10 and endoframe 18 relative to inner shaft 20 during delivery and positioning at the aneurysm.

Tether 42 may comprise a tether loop fixedly attached to inner shaft 20 distal of the filling structure 10, the tether loop extending through an opening in endoframe 18 and around release wire 46, as shown in detail in FIG. 14E. Tether 42 couples the endoframe 18 to inner shaft 20 so long as release wire 46 remains within the loop of tether 42. In this embodiment, the filling structure 10 is indirectly coupled to inner shaft 20, since endoframe 18 is also coupled to the filling structure 10 with sutures 47. Once release wire 46 is withdrawn, tether loop 46 easily slips out through the opening in endoframe 18 and can be withdrawn with inner shaft 20.

Tether 44 may comprise a tether loop fixedly attached to filling tube 26, which then extends through a suture loop 45 on the proximal end of filling structure 10 and around the release wire 46. So long as release wire 46 remains within the suture loop 45, filling tube 26 remains coupled with the proximal end of filling structure 10. Tether 44 may inhibit movement of the proximal region of the filling structure 10 during delivery, and may help prevent release of the fill tube 26 from the filing structure 10. Thus, tether 44 provides a fail safe mechanism prior to filling and during filling or re-filling of the filling structure, until the procedure is over, at which time the release wire can be retracted releasing the coupling between filling tube 26 and the filling structure 10.

Once the filling structure 10 has been deployed and filled with hardenable fluid filling medium, the release wire 46 can be withdrawn by pulling the proximal end of the release wire 46 from the handle 60. After releasing the filing structure 10 and endoframe 18, the delivery system can be withdrawn from the body as tether loops 42 and 44 no longer couple the filling structure 10 or endoframe 18 to the system. In an exemplary embodiment, the release wire 46 extends the length of and runs parallel to inner shaft 20. During delivery, the distal end of release wire 46 is releasably coupled to inner shaft 20 just proximal of the nosecone 30, as shown in detail in FIG. 14D. This is to ensure the release wire does not interfere with deployment of the filling structure 10. A UV adhesive, such as Loctite 3321, may be applied to hold the knot and create a temporary bond between the release wire and the inner shaft. In many embodiments, release wire 46 is Teflon coated, so that the temporary bond easily delaminates and releases from inner shaft 20 when a physician pulls the proximal end of release wire 46 at handle 60. The release wire 46 may be made from polytetrafluororoethylene coated 304 stainless steel and sized so as to be easily withdrawn from between the filling structure 10 and the endoframe 18 after deployment.

As shown in FIG. 14B, filling structure 10 is coupled with the distal end of the endoframe 18 by one or more sutures 47, preferably four sutures, disposed at regular intervals about the distal end of filling structure 10. The sutures 47 couple the filling structure 10 to endoframe 18 to maintain the relative position of the endoframe 18 during deployment and may also help anchor the filling structure 10 to the endoframe 18 after deployment of the filling structure 10 in the aneurysm. One of skill in the art will appreciate that other releasable coupling mechanisms, including releasable knots, may be used and therefore the coupling mechanism is not limited to tether embodiments. Additionally, the tether may be used as a releasable coupling mechanism in any of the embodiments disclosed in this specification.

In an exemplary embodiment, suture 47 comprises a single thread having one end attached to endoframe 18 and the other end attached to endoframe 18, as shown in FIG. 6A. Alternatively, suture 47 may comprise a suture loop, such as those depicted in FIG. 6B. Endoframe 18 may include eyelets 49 near the proximal or distal ends of the endoframe through which the suture 47 can be looped to secure the filling structure 10 to the endoframe 18. This way, the filling structure 10 will be fixed relative to the endoframe as long as the tether loops are taut. Generally, this coupling mechanism will allow about ±5 mm and more preferably ±3 mm of relative movement between the filling structure and the endoframe. Also, the filling structure and endoframe should be positionable within ±7 mm and more preferably between ±5 mm of a target position within the aneurysm of the filling structure 10. In an exemplary embodiment, the four sutures are equally distributed about the distal end of the filling structure. For example, the four sutures may be attached to the filling structure and spaced apart radially at 0 degrees, 90 degrees, 180 degrees and 270 degrees.

While the above is a complete description of the preferred embodiments of the invention, various alternatives, modifications, and equivalents may be used. The various features of the embodiments disclosed herein may be combined or substituted with one another. Therefore, the above description should not be taken as limiting in scope of the invention which is defined by the appended claims.

What is claimed is:

1. A system for treating an aneurysm, said system comprising:
an elongate flexible inner shaft having a proximal region and a distal region, wherein the inner shaft further comprises a guidewire lumen extending therethrough;
a distally tapered nosecone disposed at the distal region of the inner shaft, the nosecone comprising a through lumen in fluid communication with the guidewire lumen, wherein the through lumen extends along a longitudinal axis of the nosecone from a proximal opening to a distal opening of the nosecone, and wherein the nosecone further comprises a series of side ports in fluid communication with the through lumen, the side ports disposed between the proximal and distal openings of the through lumen;
a guidewire disposed within the inner shaft and extending through the single through lumen of the nosecone; and
a contrast media for injecting into the single guidewire lumen and the through lumen of the nosecone, wherein the guidewire lumen of the inner shaft is sized to allow a flow of the contrast media sufficient for performing angiography while the guidewire is disposed therein.

2. The system of claim 1, wherein the single through lumen of the nosecone comprises a narrowed distal region having a diameter smaller than a diameter of the through lumen proximal of the narrowed proximal region,
wherein the through lumen is sized to simultaneously receive the guidewire and the flow of contrast media; and
wherein the narrowed distal region fittingly receives the guidewire so as to inhibit the flow of the contrast media through the narrowed distal region so as to direct the flow of contrast media out the side ports of the nosecone.

3. The system of claim 1, wherein the series of side ports comprise a plurality of pairs of side ports, the pairs of side ports arranged in a helical pattern along a longitudinal axis of the nosecone.

4. The system of claim 1, wherein the majority of side ports of the plurality are substantially perpendicular to the single through lumen of the nosecone.

5. The system of claim 3, wherein a pair of side ports comprises two side ports adjacent to one another along the longitudinal axis of the nosecone.

6. The system of claim 3, wherein the series of side ports are disposed along nosecone at regular intervals along the helical pattern.

7. The system of claim 1, further comprising:
an elongate flexible outer sheath slidably disposed over the inner shaft, wherein the distal end of the outer sheath releasably couples with the nosecone at an interface, and the distal end of the outer sheath releasably couples with a proximal portion of the nosecone and a portion of the proximal region of the nosecone is isodiametric with the outer shaft adjacent the interface.

8. The system of claim 1, further comprising:
an expandable member disposed on a distal region of the inner shaft; and
a support endoframe disposed over the expandable member; and
an endograft disposed over the support endoframe.

9. The system of claim 7, further comprising:
a first double-walled filling structure disposed over the distal region of the inner shaft, the filling structure having an outer wall and an inner wall, wherein the filling structure is adapted to be filled with a hardenable fluid filing medium so that the outer wall conforms to an inside surface of the aneurysm and the inner wall forms a first generally tubular lumen to provide a path for blood flow; and
at least a first expandable endoframe disposed within the filling structure, the first endoframe radially expandable within at least a portion of the tubular lumen of the filling structure, wherein the outer sheath is slidably disposed over the filling structure, endoframe and inner shaft;
a filling tube having a lumen fluidly coupled with and adapted to fill the filling structure with the filling medium; and
an inflation device fluidly coupled with the filling structure.

10. A method of treating an aneurysm, said method comprising:
providing an elongate flexible inner shaft having a guidewire lumen, wherein the inner shaft comprises a proximal region and a distal region, the distal region having a tapered fenestrated nosecone, the nosecone having a through lumen extending from a proximal opening to a distal opening of the nosecone;
advancing the nosecone through a patient's vasculature along a guidewire disposed within the guidewire lumen of the inner shaft and the through lumen of the nosecone so as to position the nosecone in a target region; and
releasing a contrast media into the vasculature through a series of side ports in the nosecone by injecting the contrast media through the guidewire lumen at the proximal end of the inner shaft while the guidewire is disposed in the guidewire lumen and through lumen.

11. The method of claim 10, wherein the series of side ports comprise pairs of side ports, the pairs arrange in a helical pattern along a longitudinal axis of the nosecone, and the method further comprises:
imaging a flow of contrast media through the aneurysm with fluoroscopy

12. The method of claim 10, wherein the inner shaft further comprises a first double-walled filling structure attached to a first endoframe, at least a portion of which is disposed within the first double-walled filling structure, the endoframe and/or filling structure being removably attached to the distal region of the inner shaft, the method further comprising:
advancing the inner shaft in the vasculature such that the first double-walled filling structure traverses the aneurysm;
radially expanding the first endoframe from a contracted configuration to an expanded configuration; and
filling the first filling structure with a hardenable fluid filling medium so that an outer wall of the first filling structure conforms to an inside surface of the aneurysm and an inner wall of the first filling structure forms a first substantially tubular lumen to provide a first blood flow path across the aneurysm, and wherein the at least a portion of first endoframe is disposed in the first substantially tubular lumen.

13. The method of claim 12, further comprising:
pre-filling the first filling structure with saline or contrast media so that an outer wall of the first filling structure conforms to an inside surface of the aneurysm and an inner wall of the first filling structure forms a first substantially tubular lumen to provide a first blood flow path across the aneurysm, and wherein the at least a portion of first endoframe is disposed in the first substantially tubular lumen, wherein releasing the contrast media is performed after the pre-filling;
imaging the flow of the contrast media using fluoroscopy to detect the presence of endoleaks around the pre-filled filling structure;
adjusting a filling pressure or volume of the pre-filled filling structure until no endoleaks are observed during fluoroscopy; and
recording the filling pressure and/or volume of the prefilled filling structure at which fluoroscopy indicated that no endoleaks were observed,
wherein filling with the hardenable fluid filling medium comprises filling the filling structure at the recorded filling pressure and/or filling volume.

14. The method of claim 13, further comprising:
releasing a contrast media into the vasculature through a series of side ports in the nosecone by injecting the contrast media through the guidewire lumen after filling the filling structure with the hardenable fluid filling medium;
imaging the flow of the contrast media using fluoroscopy to detect the presence of endoleaks around the filling structure filled with the hardenable fluid filling medium; and
adjusting a filling pressure or volume of the pre-filled filling structure until no endoleaks are observed during fluoroscopy.

15. The method of claim 12, wherein releasing the contrast media is performed before, during or after filling of the filling structure with the hardenable fluid filling medium.

16. The method of claim 12, further comprising:
releasing the first filling structure and first endoframe from the inner shaft by retracting a release wire thereby disengaging a coupling mechanism attaching the filling structure and endoframe to the inner shaft.

17. The method of claim 12, further comprising:
providing a second elongate flexible inner shaft having a proximal end, a distal end, and a second expandable member near the distal end, the second flexible inner shaft carrying a second radially expandable endoframe over the second expandable member and a second double walled filling structure disposed over the second endoframe;
advancing the second shaft in the vasculature of the patient so that the second filling structure is delivered to the aneurysm;
filling the second filling structure with a second hardenable fluid filling medium so that an outer wall of the second filling structure conforms to an inside surface of the aneurysm and to the first double-walled filling structure, and an inner wall of the second filling structure forms a second substantially tubular lumen to provide a second blood flow path across the aneurysm;
radially expanding the second endoframe from a contracted configuration to an expanded configuration, wherein in the expanded configuration the second endoframe engages the inner wall of the second filling structure;
hardening the second fluid filling medium in the second filling structure; and
releasing the second filling structure from the second flexible shaft.

18. The method of claim 17, wherein releasing the contrast media comprises releasing the contrast media into the vasculature before, during or after filling or pre-filling of the first or second filling structure so as to observe a flow of fluid through the vasculature with fluoroscopy.

19. A method for constructing a retractable catheter shaft, said method comprising:
providing an outer sheath having a distal and a proximal end and a hypotube having a proximal and a distal end, the outer sheath having an inside diameter larger than an outside diameter of the hypotube;
covering an outside surface of the proximal end of the outer sheath in a laminate material;
providing an annular knob, the annular knob having a tubular strain relief member extending from one side, the strain relief member having an inside diameter larger than an outside diameter of the outer sheath;
slidably interfacing the tubular strain relief member with the proximal end of the laminate covered outer sheath so as to slidably receive the outer sheath within the tubular strain relief member;
heating the interfaced tubular strain relief member and outer sheath to a temperature sufficient for the laminate to bond with the outside surface of the outer sheath and/or the knob; and
inserting the distal end of the hypotube through the annular knob from a side opposite the tubular strain relief member so that the hypotube extends through the annular knob and is slidably received inside the outer sheath such that pulling the annular knob proximally slidably retracts the outer sheath in the proximal direction over the hypotube.

20. The method of claim 19, wherein the distal end of the hypotube and/or the proximal end of the outer sheath comprises a retaining mechanism to prevent the hypotube and outer sheath from axially separating after the hypotube is inserted into the outer sheath.
